(19) United States
(12) Patent Application Publication
(10) Pub. No.: US 2004/0053885 A1
(43) Pub. Date: Mar. 18, 2004

(54) Use of hyaluronic acid derivatives for the prevention of inflammatory arthritis

(76) Inventors: Rudolf Venbrocks, Hainspitz (DE); Andreas Roth, Eisenberg (DE); Peter-Juergen Mueller, Jena (DE); Stephanie Moeller, Jena (DE); Joerg Ozegowski, Jena (DE); Gundela Peschel, Jena (DE)

Correspondence Address: JACOBSON HOLMAN PLLC, 400 SEVENTH STREET N.W., SUITE 600, WASHINGTON, DC, 20004, US

(21) Appl. No.: 10/399,710

(22) PCT Filed: Oct. 19, 2001
(86) PCT No.: PCT/DE01/03984

(30) Foreign Application Priority Data
Oct. 19, 2000 (DE) 100 53 053.2

(51) Int. Cl. A61K 31/728

(52) U.S. Cl. 514/54

(57) ABSTRACT

Rheumatoid arthritis is a chronic inflammatory disease, leading to joint destruction. Conventional therapy is based on pain-reduction and an improvement in the frictional properties of joints, in order to delay the time for operative intervention. A lack of specifically-acting agents for drug-based therapy for arthritis exists. The formulations comprise sulphated hyaluronic acids with varying degrees of sulphation, or the pharmacologically acceptable salts thereof and, optionally, hyaluronic acid and/or hyaluronic acid uronide. The pharmaceutical formulations are highly concentrated injection preparations with an aqueous, viscous, gel-like, or paste-like form, or a low-concentration rinsing fluid for intra-articular application.

The present invention relates to novel formulations having effectiveness against rheumatoid arthritis (RA). RA is a chronic inflammatory disease which goes through several stages and finally results in a massive destruction of joints or inflammations in the tendon area. According to the recent state of knowledge, it is considered that T cells initiate and maintain the inflammation. In this process, cytokines as well as mesenchymal cells (macrophages and synovial fibroblasts) are active. It is very probable that the cytokine TNF-α is one of the mediators of the inflammation. This cytokine is mainly released by the macrophages. It promotes the formation of the pannus, which is typical of RA and promotes cartilage destruction. TNF-α increases the number of adhesion molecules for leucocytes on the surface of the endothelial cells and the fenestration of the capillary endothelial layer. This results in an increased inflow of leucocytes into the synovia. The cytokine promotes the secretion of matrix metalloproteinases by the synoviocytes. These enzymes are directly involved in the destruction of bones and cartilage. TNF-α also sensitizes pain receptors, which is associated with the induction of pain sensations. TNF-α plays a critical role in the initiation and maintenance of rheumatic arthritis.

Hyaluronic acid is formed both by animals as a component of the synovial fluid of the joints and other tissues, e.g., the vitreous body of the eye, and by bacteria of the genus Streptococcus. Due to its viscoelastic properties, it increases the slip of the joints and acts as a shock absorber. It forms viscoelastic solutions.

Hyaluronic acid is an endogenous glucosaminoglycan which contains repeats of a disaccharide consisting of D-glucuronic acid and N-acetyl-D-glucosamine. Each disaccharide is connected to the next through a β-(1-4) linkage. This bond can be cleaved hydrolytically by the enzyme hyaluronidase, which is also endogenous. There is an equilibrium of anabolism and catabolism (turnover) of hyaluronic acid in the body. Under the influence of free-radicals which cause inflammations, hyaluronic acid is successively degraded, during which its viscoelastic properties decrease. Thus, in the course of HA, the position of the turnover equilibrium of endogenous hyaluronic acid is disturbed. Uronides of hyaluronic acid are prepared by the action of the enzyme hyaluronate-lyase on hyaluronic acid (WO 00/38647).

According to the state of the art, medicaments having a symptom-modifying effect and substances having a chondroprotective or structure-modifying effect are distinguished. For the medicamental treatment of RA, a large number of orally and subcutaneously administered steroid and non-steroid antirheumatics and a group of medicaments which have a high similarity with the synovial fluid or building materials of cartilage and are administered intraarticularly (i.art.) are employed. The steroidal antirheumatic agents have a systemic effect as anti-inflammatory agents.

Orally administered medicaments include, for example, methotrexate (Lantarel), a purine antagonist and cytostatic agent, and leflunomide (Arava), a pyrimidine antagonist and immunosuppressant. Subcutaneously administered medicaments include, for example, Etanercept (Enbrel), a TNF-α inhibitor. Other pharmaceuticals include hydroxychloroquine sulfate, gold (Tauredon) and penicillamine (Trisorcin).

Caruso et al. (U.S. Pat. No. 4,312,866) describes a therapy for the treatment of RA with the antibiotic Rifamycin SV in combination with basic amino acids.

Chondroprotective agents include, for example, mixtures of mucopolysaccharide polysulfate esters of animal origin which can be employed in both degenerative arthrosis and RA. A polysulfated glycosaminoglycan is employed under the designation of Arteparon or Adequan RTM for use in animals (Luitpold Pharmaceuticals) with molecular weights of between 3,000 and 17,000 D. The polysulfated glycosaminoglycans promote the formation of hyaluronic acid at the synovial membrane (Nishikawa et al. in “Influence of sulfated glycosaminoglycans on the biosynthesis of hyaluronic acid in rabbit knee synovial membrane”, Arch. Biochem.

Biophys., 240, 146-153). The monomeric glucosamine sulfate (Dona, Rottapharm) is also employed as an agent for treating arthrosis.

Sulfated hyaluronic acid or sulfated dextran have anti-inflammatory properties with systemic activity (JP 8.277.224). It is suggested to inject it intravenously in amounts of from 0.1 to 10 mg/kg of body weight, for example, in dyspnea, in the form of a solution which contains from 0.5 to 10 mg/ml of sulfated polymers.

Sulfated hyaluronic acid is further known for its heparin-like anticoagulant and antithrombolytic and anti-inflammatory properties. In addition, effects of reducing cell adhesion are described.

Lanfranco et al. (WO 98/45335) and WO 99/43728) employ sulfated hyaluronic acid in pharmaceutical formulations and biomaterials and for the coating of biomedical objects.

Cialdi et al. (U.S. Pat. No. 6,027,741) describe sulfated polysaccharides, for example, sulfated hyaluronic acid and its esters, as having anticoagulant and cell-adhesion reducing properties for use in biomaterials.

Pure hyaluronic acid, which has a good effect (U.S. Pat. No. 4,808,576) in degenerative arthrosis, belongs to the slowly acting medicaments, displaying an effect only after 3-5 injections, wherein a chondroprotective effect can be observed. This effect has not been demonstrated in therapies of RA as yet, so that unmodified hyaluronic acid is hardly employed in RA. Thus, the commercially available injection preparations with hyaluronic acid having an average molecular weight of between 500,000 D and 900,000 D have no effect in the clinical picture of RA.

A practiced method for the treatment of RA is the intra-arterial injection of injection preparations with hyaluronic acid (Lindblad, U.S. Pat. No. 4,801,619) containing additives with anti-inflammatory properties. These additives are, for example, steroids, such as prednisolone, dexamethasone, but also ibuprofen (antirheumatic agent) or sulfated mucopolysaccharides as a by-product in the hyaluronic acid prepared from animal material (Drizen et al., U.S. Pat. No. 5,079,236). A disadvantage of the proposed sterile formulations for veterinary applications in U.S. Pat. No. 5,079,236 is the fact that sterility is achieved by the addition of preservatives, such as the cell-toxic parabenes. The stated amounts of sulfated mucopolysaccharides are between 0.75 and 1.25%, based on the stated 5 to 20 mg of sodium hyalunorate per ml of injection solution, i.e., between 0.037 and 0.25 mg/ml of sulfated mucopolysaccharides is contained in the preparation as an impurity.

According to the state of the relevant art, there is a deficiency in topically applied medicaments having a specific effect against the different stages of development and manifestations of the clinical picture of rheumatoid arthritis.

It is the object of the present invention to propose such specifically effective and topically acting therapeutic formulations for the well-aimed treatment of rheumatoid arthritis. According to this object, the formulations should be well tolerated, of non-animal origin and have a defined chemical composition if possible.

The invention relates to the use of sulfated hyaluronic acids, especially in the form of isotonic and sterile formulations, for intraarticular application and applications in the tendon area. With a surprising healing success, such formulations are employed for the treatment of inflammatory and degenerative joint and tendon diseases in the initial, acute and chronic stages. According to the invention, they can be administered as a more highly concentrated injection preparation and remain in the body, or employed as a less concentrated rinsing liquid. In the studies on which the invention is based, a very clearly improved effect in the treatment of rheumatoid arthritis was found as compared with the per se known injection or rinsing with isotonic hyaluronic acid solutions.

The use of sulfated hyaluronic acids according to the invention is effected in aqueous solution, especially in isotonic aqueous solutions which contain therapeutically effective doses of sulfated hyaluronic acid with an application-specific degree of sulfatation. Such solutions are injected into the intraarticular cavity of a joint or into the tendon sheath of in the environment of tendons and remain there. When employed as a rinsing liquid, the intraarticular cavity or the environment of a tendon is rinsed with the formulations, and the active substance does not remain at the site of inflammation. In another embodiment, higher viscosity formulations containing the sulfated hyaluronic acids are also employed as an injection preparation for the intraarticular cavity. In other embodiments according to the invention, highly viscous solutions are injected, or the gels or pastes according to the invention are administered into the joint capsule of in the environment of the tendon, optionally with application of increased pressure in the injection device, through a cannula or injection needle. The application of the highly viscous or gel-like or paste-like formulations is advantageous in every case where the formation of a depot is desirable.

In another embodiment of the invention, the highly viscous to paste-like formulations employed in the use according to the invention contain a viscosity-increasing or gel-forming hydrocolloid, preferably hyaluronic acid, as an auxiliary agent, in addition to the sulfated hyaluronic acid. As a rule, the hyaluronic acid or other hydrocolloids cause a higher viscosity or gel structure of the formulations. An advantage of employing hyaluronic acid in the formulations is the fact that hyaluronic acid additionally causes a chondroprotective effect. In addition to hyaluronic acid, for example, other polyanionic polysaccharides, such as xanthan, alginic acid or pectic acid, may be employed.

In another embodiment, the uronide of hyaluronic acid may also be employed to advantage instead of hyaluronic acid. The uronide is prepared in a per se known manner by the enzymatic cleavage of hyaluronic acid with the microbial enzyme hyaluronate-lyase. The uronide contains unsaturated bonds at the terminal glucuronic acid residues. Due to its generally lower molecular weight, the uronide contributes less to the increase of viscosity. An advantage is its particularly high radical-binding property, which goes beyond the effect of hyaluronic acid. The inflammatory processes in the joints or in the environment of the tendons are weakened even more intensively, and the healing effect of the formulations is enhanced.

The highly viscous, paste-like or gel-like formulations are prepared, for example, in the following way. From a sterile-filtered liquid formulation which optionally contains a further hydrocolloid, e.g., hyaluronic acid or hyaluronic acid uronide, the water is withdrawn, for example, by freeze-drying under sterile conditions. This is followed by the addition of isotonically adjusted sterile water in an amount which results in a highly-viscous to paste-like formulation.

The injection solutions contain between 1.0 mg/ml and 200.0 mg/ml, preferably between 10.0 mg/ml and 50.0 mg/ml, of sulfated hyaluronic acid.

In the rinsing solutions employed according to the invention, the concentration of sulfated hyaluronic acid is between 0.01 mg/ml and 20 mg/ml. In one embodiment, preferably in applications for rheumatoid arthritis which is less massively manifested, the degree of sulfatation of the hyaluronic acid is within a range of from 0.1 to 2.0, based on a disaccharide unit, whose maximum degree of sulfatation may be 4.0. In another embodiment, which is preferably employed in massive clinical pictures of rheumatoid arthritis, the degree of sulfatation of the sulfated hyaluronic acid employed according to the invention is within a range of from 2.0 to 4.0.

The molecular weights of the sulfated hyaluronic acids according to the invention are between 1,000 and 500,000 D. The isotonic property of the aqueous formulations is caused by a content of inorganic salts, preferably common salt.

To support the healing effect in the use of sulfated hyaluronic acids according to the invention, another active ingredient, such as an antibiotic or an additional anti-inflammatory substance, for example, a cyclooxygenase inhibitor, may optionally be added to the formulation.

Without limiting the invention thereby, some typical fields of application of the invention may be described in which a surprising inhibition of inflammatory arthritis was detected.

As an injection preparation employed according to the invention, the formulation is injected intraarticularly into the joint cavity, into the small vertebral joints and into the sacroiliac joint in rheumatic diseases. In tenosynovitis of rheumatic and idiopathic origins, injection directly into tendon sheathes or into the wider environment of the tendons proved successful. Intraarticular injections may also be employed after arthroscopic interventions in all large and small joints in which an inflammatory component of the joint mucosa can be seen.

Employing the formulation in arthroses of the small and large joints (e.g., coxarthroses, gonarthroses, omarthroses) in the inflammatory stage also proved to be favorable to the treatment.

In another embodiment, rinsing solutions according to the invention are preferably employed for the postsurgical rinsing of large and small joints treated by arthroscopy, with endoprostheses or openly by means of synovectomy, in which an inflammatory component of the joint mucosa can be seen (e.g., rheumatoid arthritis, reactive arthritis or activated arthrosis).

Due to the different degrees of sulfatation of the sulfated hyaluronic acid employed according to the invention, there are formulations which are adapted to different kinds of treatment. The higher the degree of sulfatation, the more intensive is the specific effect of the sulfated hyaluronic acid, while the chondroprotective properties or the hyaluronic-acid specific properties are reduced. The intensity of the treatment can be varied in accordance with the clinical picture. For rinsing, the active substance can be employed with a lesser degree of sulfatation, which also holds for less severe diseases or the preliminary of the disease. In severe cases of inflammation, highly sulfated hyaluronic acid is preferably employed, while later, when the manifestations have subsided, formulations which contain less sulfated hyaluronic acid and optionally hyaluronic acid are also employed.

In addition to the unexpected very intensive effect and the very high tolerability even of high doses of sulfated hyaluronic acid in the formulations, other properties of sulfated hyaluronic acid are also advantageous in comparison with solutions of pure hyaluronic acid.

Thus, it is advantageous that the solutions of sulfated hyaluronic acid generally have a lower molecular weight and thus a lower viscosity as compared to equally concentrated solutions of pure hyaluronic acid. Therefore, sulfated hyaluronic acid can be injected in higher concentrations and/or in smaller volumes. Also, the formulation can be employed in a sterile-filtered form.

As compared to the other active substances, sulfated hyaluronic acid is a derivative of human-identical hyaluronic acid. It can be assumed that the detected high tolerability of the active substance is related to the structural closeness of the active substance according to the invention with native hyaluronic acid or with the sulfated glycosaminoglycans. Another great advantage arises from the thrombolytic properties of sulfated hyaluronic acid. This simultaneously prevents the formation of thrombi after injury of blood vessels in and around the joint by the injection.

The anti-inflammatory effect in the joint and tendon area appears to be a specific effect of sulfated hyaluronic acid. This surprising effect, which goes beyond a general anti-inflammatory action, was neither predictable nor previously known for the applications of the formulations according to the invention in the joint and tendon area.

The high capacity of the formulations according to the invention containing sulfated hyaluronic acid for the specific treatment of arthritis and in the protection of cartilage as compared to unsulfated hyaluronic acid is demonstrated in vivo by an experimental examination with animals (Example 2).

The importance of the invention resides in the use of the sulfated hyaluronic acid in the treatment of arthritis and in its influence not only on the symptomatic result of detumescence of the joints, but also the inhibition of cartilage and bone destruction in all stages of the disease. The application of the described hyaluronic acid preparations has been limited to the treatment of patients suffering from arthrosis which is slowly degenerative and has a predominantly non-inflammatory course. Therefore, the use of the formulations according to the invention is of especially great importance to the very large number of patients who suffer from rheumatoid arthritis.

### EXAMPLE 1

### Preparation of the Sterile Pyrogen-free Injection Solution

A pyrogen-free sterile-filtered hyaluronic acid from Streptococcus equisimilis having a defined molecular weight of 100,000 to 3,000,000 Dalton was used. The molecular weight was determined by size exclusion chromatography (SEC)/multi angle laser light spectrometry (MALLS). Sulfated hyaluronic acid was obtained by the sulfatation of high molecular weight hyaluronic acid in accordance with DE 19.813.234 A 1, and its molecular weight was determined by a light-scattering method (SEC-MALLS).

A 1% hyaluronic acid solution is dialyzed against pyrogen-free water until the conductivity of the water has sunken below 20 mS. The solution is lyophilized. Two liters of 0.2% hyaluronic acid is treated with active charcoal and filtered through a 1 to 2 cm thick layer of silica gel of the type “Köstrosorb” (Chemiewerk Bad Köstritz GmbH, Bad Köstritz) as a filtering aid. Thereafter, filtration is effected through a 0.8 μm and subsequently through a 0.2 μm cellulose acetate filter.

The hyaluronic acid, which is now pyrogen-free, is lyophilized under sterile conditions, and then sterile physiological saline is added.

For comparative purposes, a 1% solution of a high molecular weight hyaluronic acid or their salts in physiological NaCl solution is prepared and sterile-filtered at room temperature.

### EXAMPLE 2

### Examination of the Anti-inflammatory Effect on the Knee Joint in Rats

The animal model employed, antigen-induced arthritis of rats, is a well established animal model which reflects the mechanisms of rheumatoid arthritis (RA) very well. The histomorphometric examinations on the untreated knee joint at the transition between cartilage and bone showed typical alterations which are not found in mice in this form due to their relatively strongly developed subchondral lamella.

Female Wistar rats are employed for this experiment. At an interval of one week, the animals are subcutaneously preimmunized with 0.1 mg of methylalbumin (mBSA) in Freund's complete adjuvant. Two weeks after the second immunization, 0.1 mg of mBSA is injected intraarticularly into the left knee joint. This injection induces arthritis, which persists for weeks after an acute phase. One day after the induction of the arthritis, the treatment is begun with sulfated hyaluronic acid solution or with high molecular weight hyaluronic acid (MW=1,800 kD), which are injected intraarticularly into the knee joint.

Parameters which describe the influence of microbially obtained high molecular weight hyaluronic acid and sulfated hyaluronic acid on the antigen-induced arthritis are examined:

1. Local evidence of arthritis (in the course of the experiment)

2. Histological arthritis score

3. Formation and morphology of microfractures in the region of the calcified cartilage

4. Measurement of lateral joint diameter

5. Biochemical parameters

The course of the experiment was as shown in Table 1

TABLE 1

| | Course of experiment | | | | | | | |
|---|---|---|---|---|---|---|---|---|
| | −3 | −2 | −1 | | +1 | +1 | +2 | +3 |
| | weeks | weeks | week | 0 | day | week | weeks | weeks |
| Immunization 1 | E 1/2 | | | | | | | |
| | C 1/2 | | | | | | | |
| Immunization 2 | | E 1/2 | | | | | | |
| | | C 1/2 | | | | | | |
| Induction of | | | | E 1/2 | | | | |
| arthritis | | | | C 1/2 | | | | |
| Sulfated hyaluronic | | | | | E 1/2 | E 2 | E 2 | E 2 |
| acid | | | | | | | | |
| Sacrifice of animal | | | | | | E 1, C 1 | | E 2, C 2 |

1 = short term
2 = long term
E = experimental group (20 animals each)
C = Control group (20 animals each)

For each intervention, the animals were anesthetized by ether anesthesia.

The immunization of the animals was effected at an interval of 7 days. In the first immunization, a total of four subcutaneous injections of 0.25 ml each (corresponding to 0.1 mg of methylalbumin in Freund's complete adjuvant) were effected on both sides of the vertebral column at the level of the shoulder blade and above the hip (each at about 1.5 cm distance from the spines of the vertebrae). In the 2nd immunization, 3 depots of 0.3 ml of solution each were introduced; one exactly between the two shoulder blades and two at the level of the abdomen on both sides paravertebrally.

The induction of arthritis was effected two weeks after the 2nd immunization by intraarterial injection of 0.05 ml each of a 10 mg/ml solution of mBSA in physiological saline into the respective right knee joint.

On the day after induction of arthritis, 0.05 ml each of a formulation containing sulfated HA was injected into the right knee joint of the 20 animals of the experimental group (use of an insulin syringe, thin cannula, 0.35×40 mm). Thereafter, injection was effected once a week (long-term group).

The lateral joint diameter of all animals was measured by means of a distance meter (Matatuyo) prior to and 1, 4 and 8 days after the induction of arthritis and then every week until the experiment was completed. The animals were also weighed each time.

As soon as on the following day after induction of the arthritis, all animals exhibited a swelling of the right knee joint. Their weight did not exhibit any strong variations.

On the 7th day, 10 animals of the experimental group and 10 animals of the control group were killed. The animals were put down by ether anesthesia, and the sacrifice of the remaining animals was effected 1 week after the last injection of a formulation containing sulfated HA into the right knee joint.

Especially the long-term experiment over 3 weeks showed that a significant decrease of the joint diameters (as a measure of the inflammatory swelling) can be detected in the animals treated with sulfated HA.

TABLE 2
Joint diameter (right)
For day 0, the joint diameter is stated.
From day 0 to day 1, the increase of joint diameter
by the immunization is stated, and from day 2 to
day 29, the decrease or increase of the joint diameter
after the injection on day 1 is stated.
Short-term experiment

| | Day 0 | Day 1 | Day 4 | Day 8 |
|---|---|---|---|---|
| Sulfated HA | 867.4 | +228 | +20 | −118 |
| HA | 913.2 | +206 | +18 | −45 |

Long-term experiment

| | Day | | | | | | |
|---|---|---|---|---|---|---|---|
| | 0 | Day 1 | Day 4 | Day 8 | Day 15 | Day 22 | Day 29 |
| Sulfated | 927.6 | 1153.9 | −4 | −127 | −203 | −212 | −231 |
| HA | | +226 | | | | | |
| HA | 926.0 | 1132.9 | +27 | −97 | −140 | −144 | −134 |
| | | +206 | | | | | |
| Control | 867.4 | 1095.3 | +20 | −45 | −100 | −190 | −174 |
| | | +228 | | | | | |

measured value on day 1
difference with respect to day 0

### EXAMPLE 3

Preparation of a rinsing liquid: 1.0 g of sulfated hyaluronic acid having a molecular weight of 150,000 D and 1.0 g of hyaluronic acid uronide having an average molecular weight of 20,000 D are dissolved in one liter of physiological NaCl solution, and the solution is sterile-filtered at room temperature.

1. Use of sulfated hyaluronic acids for inhibiting inflammatory arthritis in humans and animals.

2. The use of sulfated hyaluronic acids according to claim 1, characterized in that a formulation selected from isotonic aqueous to highly viscous solutions, gels and pastes is used.

3. The use of sulfated hyaluronic acids according to claim 2, characterized in that said formulation contains a further active substance and/or one or more auxiliary agents.

4. The use of sulfated hyaluronic acids according to claim 2, characterized in that said formulation is administered into the intraarticular cavity of a joint or into the environment of a tendon, or that the intraarticular cavity is rinsed with said formulation.

5. The use of sulfated hyaluronic acids according to claim 2, characterized in that the concentration of sulfated hyaluronic acid in liquid formulations is between 1.0 mg/ml and 200.0 mg/ml, preferably between 10.0 mg/ml and 50.0 mg/ml.

6. The use of sulfated hyaluronic acids according to claim 4, characterized in that the concentration of sulfated hyaluronic acid in the rinsing liquid is between 0.01 mg/ml and 20 mg/ml.

7. The use of sulfated hyaluronic acids according to claim 4, characterized in that the degree of sulfatation of the hyaluronic acid in the injection solution is within a range of from 0.1 to 2.

8. The use of sulfated hyaluronic acids according to claim 4, characterized in that the degree of sulfatation of the hyaluronic acid in the rinsing liquid is within a range of from 2 to 4.

9. The use of sulfated hyaluronic acids according to claim 1, characterized in that the molecular weights of the sulfated hyaluronic acid is between 1,000 and 500,000.

10. The use of sulfated hyaluronic acids according to claim 2, characterized in that the formulations have a gel-like or paste-like or highly viscous structure.

11. The use of sulfated hyaluronic acids according to claim 3, characterized in that the formulations additionally contain a hydrocolloid, preferably hyaluronic acid.

12. The use of sulfated hyaluronic acids according to claim 3, characterized in that the formulations additionally contain an uronide of hyaluronic acid.

13. The use of sulfated hyaluronic acids according to claim 2, characterized in that the formulations are employed for intraarticular injection into the small vertebral joints and sacroiliac joints in rheumatic diseases.

14. The use of sulfated hyaluronic acids according to claim 2, characterized in that the formulations are employed for intraarticular injection into the environment of tendons in tenosynovitis of rheumatic and idiopathic origins.

15. The use of sulfated hyaluronic acids according to claim 2, characterized in that the formulations are employed for intraarticular injection after arthroscopic interventions at large and small joints in which an inflammatory component of the joint mucosa can be seen.

16. The use of sulfated hyaluronic acids according to claim 2, characterized in that the formulation is employed for intraarticular injection in arthroses of the small and large joints, such as coxarthroses, gonarthroses or omarthroses, in the inflammatory stage.

17. The use of sulfated hyaluronic acids according to claim 2, characterized in that the formulation is employed for intraarticular injection after arthroscopic interventions at all large and small joints in which an inflammatory component of the joint mucosa can be seen.

18. The use of sulfated hyaluronic acids according to claim 2, characterized in that the formulations are employed for rinsing and/or injection in the initial as well as acute or chronic stages in rheumatic diseases.

19. The use of sulfated hyaluronic acids according to claim 2, characterized in that the formulations are employed for the postsurgical rinsing of large and small joints treated by arthroscopy, with endoprostheses or openly by means of synovectomy, in which an inflammatory component of the joint mucosa can be seen, such as in rheumatoid arthritis, reactive arthritis or activated arthrosis.

20. The use of sulfated hyaluronic acids according to claim 2, characterized in that the formulations are employed for local injections after intervertebral disk surgery for preventing inflammatory alterations and consecutive scarred conglutinations in terms of a postnucleotomy syndrome.
